(19) United States
(12) Patent Application Publication
(10) Pub. No.: US 2014/0228627 A1
(43) Pub. Date: Aug. 14, 2014

(54) GASTRIC ELECTROMAGNETIC BAND CONTROLLED BY AUTOMATIC EATING DETECTION

(71) Applicants: Edy E. Soffer, Los Angeles, CA (US); Jeffrey Conklin, Los Angeles, CA (US)

(72) Inventors: Edy E. Soffer, Los Angeles, CA (US); Jeffrey Conklin, Los Angeles, CA (US)

(21) Appl. No.: 14/342,605

(22) PCT Filed: Aug. 31, 2012
(86) PCT No.: PCT/US2012/053548

§ 371 Date: Mar. 4, 2014

Related U.S. Application Data
(60) Provisional application No. 61/530,903, filed on Sep. 2, 2011.

(51) Int. Cl. A61F 5/00

(52) U.S. Cl. CPC A61F 5/005
USPC 600/37

(57) ABSTRACT

Systems, apparatuses, and methods for controlling a gastric band in response to the detection of eating in a subject. The band is positioned in a subject around the subject's stomach. In one method, the band is adjusted in response to changes in electrical activity of the subject's lower esophageal sphincter. Changes in electrical activity can be monitored by a monitoring unit, which can send signals to the band to open or close depending on the changes in electrical activity.

### CROSS-REFERENCE TO RELATED APPLICATIONS

This application claims the benefit of Provisional Patent Application No. 61/530,903 filed on Sep. 2, 2011, which is incorporated by reference herein.

### BACKGROUND

1. Field of Invention

This invention relates to systems and methods utilizing automatic eating detection to control a gastric magnetic band for various purposes, including the treatment of obesity.

2. Related Art

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Currently, gastric bands in use for bariatric surgery consist of silastic tubes wrapped around the stomach. They are adjustable, to be tightened or loosened by increasing or decreasing the volume of fluid within the band. All bands in current use need to be kept at a certain tightness, so as to interfere with food intake in order to achieve weight loss. However, complications, such as acid reflux and esophageal dilatation, related to band tightness can occur and require either loosening of the band with subsequent weight gain, or removal of the band. Band replacement may also be required.

### SUMMARY

In one aspect, a method for controlling a band implanted within a subject and around the subject's stomach is provided. The method includes adjusting the band in response to changes in electrical activity of the subject's lower esophageal sphincter (LES).

Another aspect of the method includes monitoring the changes in electrical activity prior to adjusting the band and/or the adjusting comprises tightening or loosening the band around the subject's stomach.

In some aspects of the method, including the embodiments described above, the band is activated when eating is indicated by the changes in electrical activity, and is deactivated at preset times after being activated. In yet another aspect, including the embodiments described above, the adjusting comprises an intervention stage, and the intervention stage comprises a start phase, hold phase and stand-by stage.

In another aspect of the method, including the embodiments described above, the band is adjusted using electromagnetic force.

In embodiments of the method, including the embodiments described above, the subject is undergoing treatment for obesity, treatment to prevent obesity, treatment for diabetes, or any combination thereof.

In some aspects, a system for a subject is provided. The system includes a monitoring unit that monitors electrical activity changes of the subject's lower esophageal sphincter; and an adjustable band that can be implanted within the subject and around the subject's stomach and that responds to signals from the monitoring unit about the electrical activity changes.

In another aspect, including embodiments of the system described above, the system has a monitoring unit that comprises a microprocessor that monitors electrical activity and detects eating and/or generates electrical signals based on the monitored electrical activity. In yet another aspect of the system, including the embodiments of the system described above, the system is individualized for each subject.

In some aspects, including the embodiments of the system described above, the system further includes one or more electrodes for detecting the electrical activity changes. The electrodes are positionable within, in contact with or proximate to the gastroesophageal junction of the subject.

In one aspect a band that can be implanted within a subject and around the subject's stomach is provided. The band can be used in embodiments of the method and/or system. The band includes two jaws hinged by a rotating axis on one side of the jaws, and two electromagnets on the other side of the jaws, one electromagnet for closing the jaws, the other electromagnet for keeping the jaws closed. Each jaw can be covered by an inflatable covering, such as an inflatable Silastic bag filled with fluid, for tightening or loosening the band around the stomach.

In another aspect, a band that can be implanted within a subject and around the subject's stomach is provided. The band can be used in embodiments of the method and/or system. The band includes a plurality of electromagnetic segments arranged end-to-end in a series, each segment comprising a rod connected to an electromagnet; and a locking electromagnetic segment connected to one or both ends of the series. When the band is relaxed and in an open status, the electromagnet of each segment in the series can be in an open position. When the band is constricted and in a closed status, the electromagnet of each segment in the series can be in a closed position. The locking electromagnetic segment can attach to both ends of the series when the band is positioned around the stomach, thus forming a closed loop around the stomach. The series of electromagnetic segments together with the locking electromagnetic segment can be covered by an inflatable covering, such as an inflatable Silastic bellow tube filled with fluid, for tightening or loosening the band around the stomach.

### BRIEF DESCRIPTION OF THE DRAWINGS

Exemplary embodiments are illustrated in referenced figures. It is intended that the embodiments and figures disclosed herein are to be considered illustrative rather than restrictive.

FIG. 1 is a schematic description of the system.

FIG. 2 is an illustration of a one embodiment of a band.

FIG. 3A-C is an illustration of another embodiment of a band.

FIG. 4 is an illustration of a representative location for detection of LES electrical activity for use in accordance with an electromagnetic band system in conjunction with automatic eating detection.

### DETAILED DESCRIPTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention.

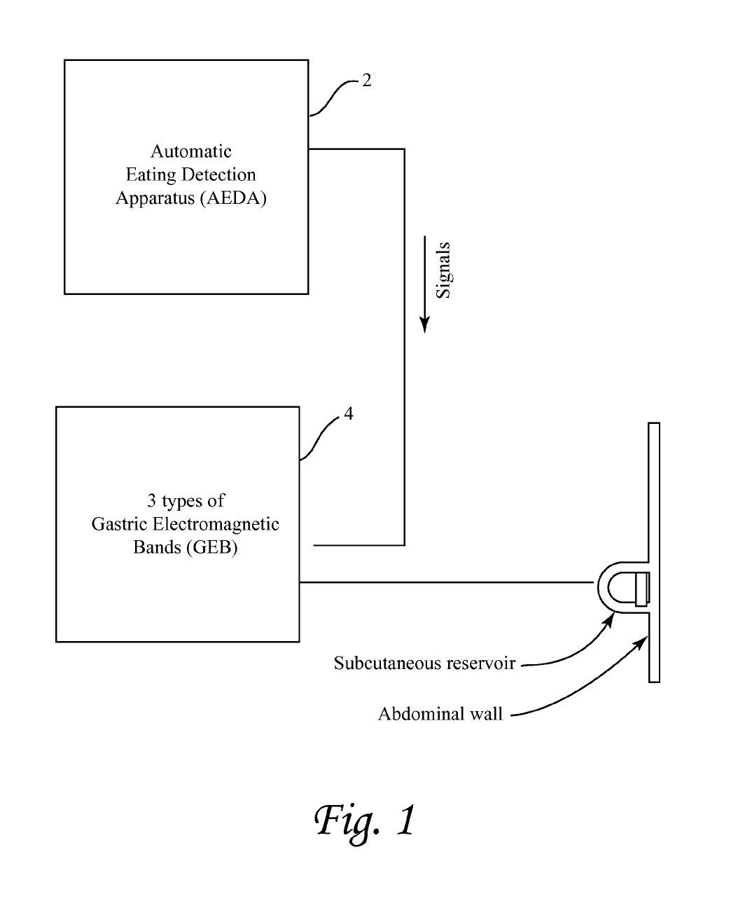

The inventors recognize that a need exists in the art for systems and methods of actively adjusting a gastric band to avoid the complications described above. Thus, in one aspect, apparatuses, systems and methods for actively adjusting a gastric magnetic band in conjunction with food intake are provided. The system function is based on two principles: the ability to automatically detect food ingestion, and the ability to mechanically constrict the stomach in order to limit food intake. The system function can comprise two mechanisms (see FIG. 1): the Automatic Eating Detection Apparatus (AEDA) that can detect food ingestion, and a Gastric Electromagnetic Band (GEB), that can constrict the stomach on demand, thus limiting food intake. The gastric electromagnetic band can be constructed in various ways, to achieve constriction by activation of electromagnetic components within the band. In this way, the system allows for constricting the stomach only when food ingestion is detected, and the gastric electromagnetic band can then be released, keeping the stomach free of any constriction for most hours of the day and during sleep. The following illustrations provide some embodiments, though not limited to these, of structures of electromagnetic systems that can be used to constrict the stomach.

Examples of subjects that can benefit from these apparatuses, systems and methods include but are not limited to obese subjects who require a bariatric surgery for weight loss. A magnetic band can be tightened at the onset of a meal, and kept at a tightened position for a defined period of time after the onset of the meal, but remain loosened most of the time. This can avoid complications and can function in a physiologic way.

Embodiments of the present invention provide for a system, comprising an adjustable gastric magnetic band, and an automatic eating detection apparatus (AEDA).

The system can be placed by laparoscopy.

FIG. 1 depicts two main components of an embodiment of the system. Upon detection of food ingestion by an automatic eating detection apparatus 2, a signal is sent to a gastric electromagnetic band system 4 to induce constriction. Constriction can then be released by a subsequent signal.

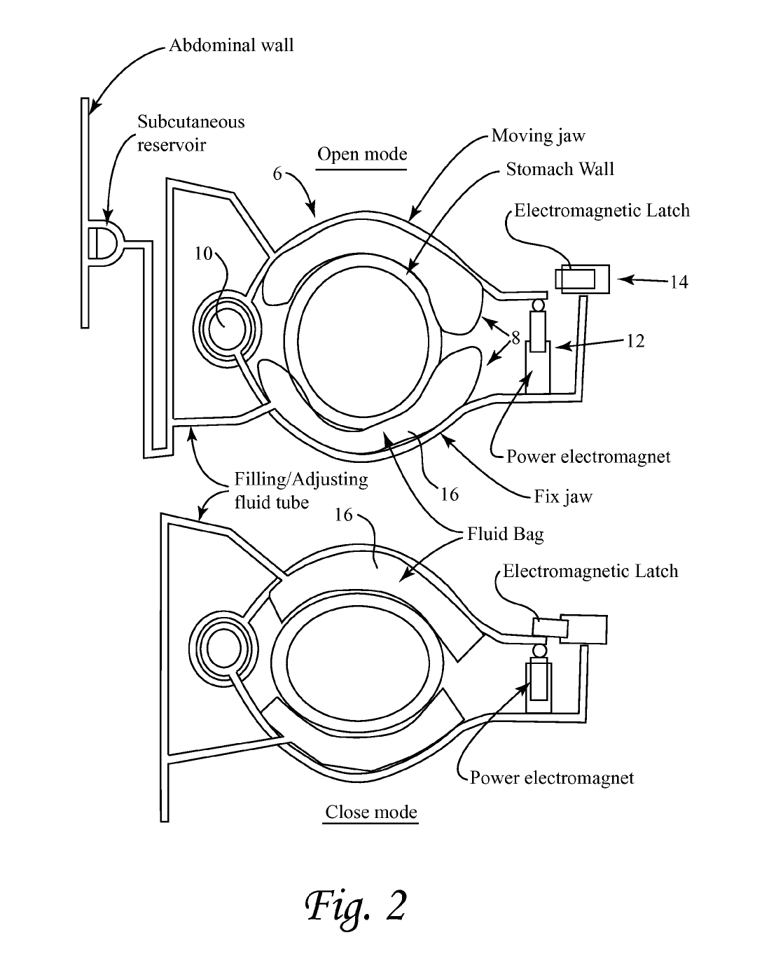

FIG. 2 depicts one embodiment of a gastric electromagnetic band. The band 6 is composed of two stiff jaws 8 hinged by a rotating axis 10 on one side and a combination of two electromagnets on the other side; one a power electromagnet 12, and the second is a locking electromagnet 14 to lock the band during the CLOSED status of the device. Upon a signal from the automatic eating detection apparatus, the system will be locked as described above, thus keeping the stomach constricted. During the CLOSED status no power is required to keep the band closed. When the computerized automatic eating detection apparatus sends an OPEN signal to the gastric electromagnetic band, the band unlocks and opens the system, and again no power is required to keep it in the OPEN position. The two stiff jaws are covered by two separate inflatable coverings such as inflatable Silastic bags 16 filled with fluid whose pressure is adjusted by the sub-system, as described in the introduction. Communication between the automatic eating detection apparatus and the gastric electromagnetic band can be achieved by way of wires. The system can be introduced by laparoscopy.

In operation, the device has two electromagnets: the power electromagnet and the latch electromagnet. The microprocessor that controls the system gives both electromagnets synchronized signals that allow the electromagnets to operate. In the CLOSED position the latch is positioned by the electromagnet, activated by the microprocessor, and positioned in such a way that it mechanically locks the movable jaw in position (see FIG. 2). So there is no need for continuous magnetic force, and hence no need for further current delivery. To OPEN the band, another set of current signals is delivered to both electromagnets. These will unlock the latch mechanism and will return the band to its OPEN position, with no further electric power requirements. The electric power requirements is needed only for a short time during the transition between the OPEN and CLOSED status.

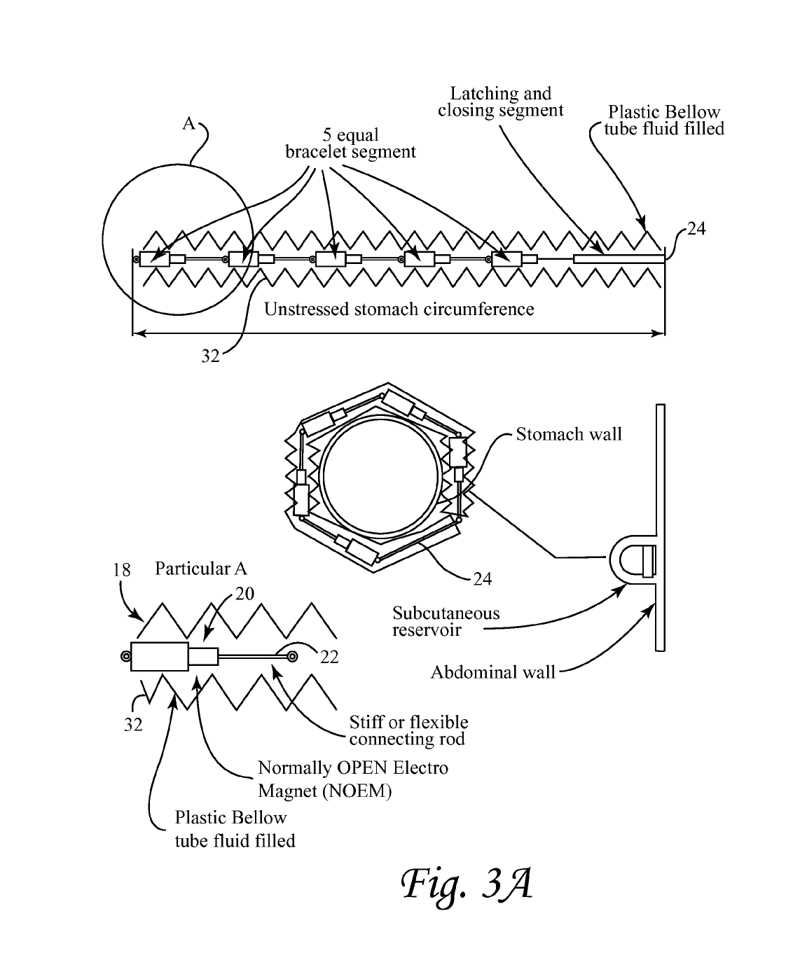

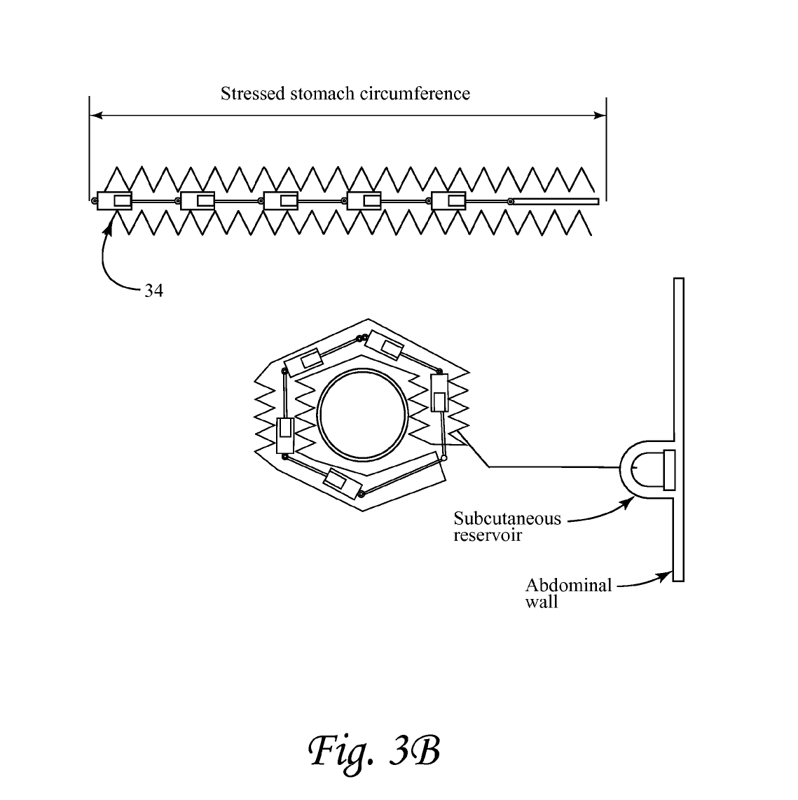

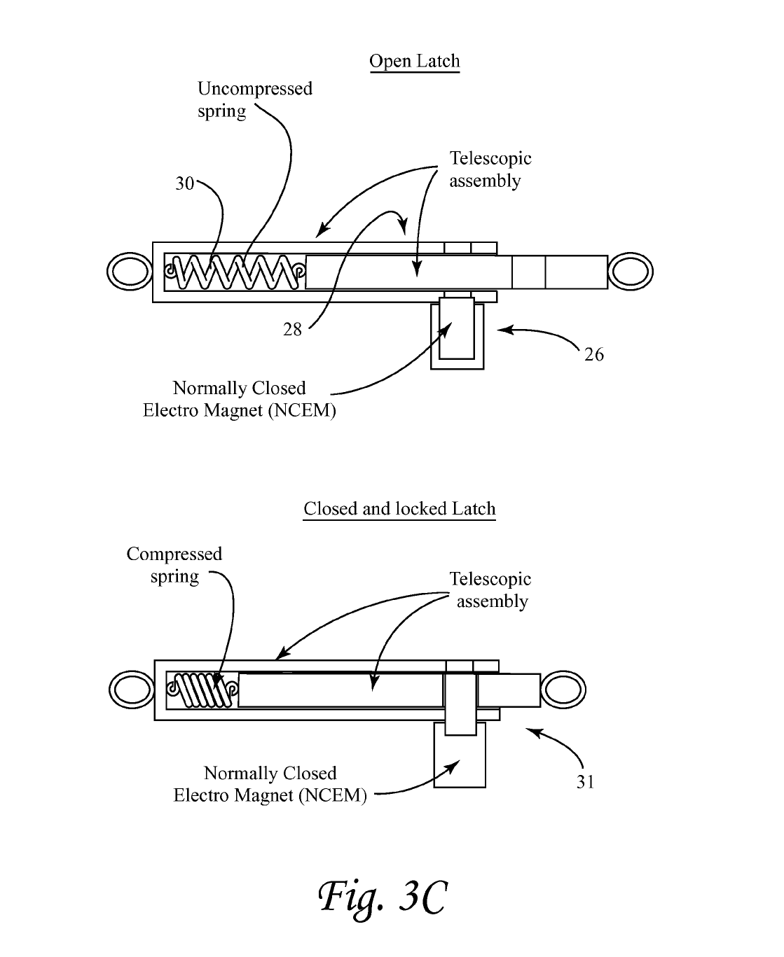

FIG. 3A-C depicts another embodiment of a gastric electromagnetic band, in this case an electromagnetic bracelet band, in open and closed status. This device comprises a chain of several electromagnetic elements (or “segments”, five in this embodiment), each segment 18 composed of a Normally Open Electro Magnet (NOEM) 20 and connecting rod 22 as shown in the expanded view of view A. Each rod is connected to an electromagnet. The rods can be manufactured from stiff or flexible materials, in turn rendering the bracelet band stiff or flexible accordingly. The sixth element (or “segment”) is a latching and locking device 24 (or “latching and closing segment”). As shown in FIG. 3C, the latching and locking device comprises a Normally Closed Electro Magnet (NCEM) 26 fitted at a 90° angle to the chain elements. The device includes a telescopic assembly 28 that includes a spring 30 at one end of the assembly. When the latching device is open, the spring is uncompressed. When the spring is compressed, the electromagnet 26 locks the assembly in a closed position. In particular, when the system is in the OPEN position, the spring is in a released status. When a current is delivered, the electromagnets are activated, producing tightening of the bracelet and consequently compression the spring, which is the sixth segment of the bracelet. A second signal activates the Latch electromagnet 26 moving it to the lock position 31 (see FIG. 3C). Once in this position, the spring remains closed by the mechanical force of the latch. The chain of six elements is introduced and sealed in an inflatable covering such as a Silastic bellow tube 32 filled with variable pressure fluid connected to the sub-system described in the introduction. The electromagnetic elements are connected to the automatic eating detection apparatus by wires, and the system can be introduced by laparoscopy.

In FIG. 3A: the band is in open status, with each electromagnet 20 in open position. In FIG. 3B, the device is in closed status with each electromagnet 34 in closed position. FIG. 3C depicts the latch in open status and locked status.

Gastric Electromagnetic Band

In various embodiments, the gastric magnetic band comprises an electromagnetic band system that can encircle the stomach when implanted within a patient, and be tightened and loosened on demand, through application of an electromagnetic force (see the embodiments in FIGS. 2 and 3). The gastric electromagnetic band can be made of any suitable material. In various embodiments, the band is made entirely of magnetic elements. In various embodiments, the gastric band is made of a synthetic material, but contains electromagnetic elements in parts of the band that serve to tighten and loosen the band around the stomach (see illustrations). In various embodiments, the material for the gastric band is a non-toxic polymer of varying firmness. Along the inner circle of the band in various embodiments runs a tube filled with fluid. The tube is connected by a thin tube to a reservoir implanted underneath the skin that can be accessed through the skin. This serves as a safety measure in case of an emergency, or in case of malfunction, when the band needs to be loosened, by removing some of the fluid. This also allows for fine adjustment of band tightness.

The band is connected to a microprocessor that detects electrical recording, process algorithms and sends signals to activate or deactivate the magnetic band. In various embodiments the band is activated when eating is detected and then deactivated at preset times after eating is detected.

Electromagnetic Band Function

A built-in algorithm for automatic eating detection controls the function of the magnetic band. The algorithm can be individualized, so as to provide the most appropriate detection for each subject. (See, Sanmiguel et al. The Effect of Eating on Lower Esophageal Sphincter Electrical Activity. Am J. Physiology 2009; 296: G793-G797; incorporated by reference herein.)

The first step is the eating detection. A change in the amplitude of electrical activity in the LES region is detected by implanted electrodes connected to an implanted microprocessor running a dedicated algorithm. The algorithm is based on the fact that the amplitude of electrical activity increases when food is consumed, and is highest with solid food compared to liquids, or swallows of saliva. This detection mechanism can be individualized and adjusted to the response of each subject. When eating is detected, a signal is transmitted to activate the electromagnetic band system, allowing the band to tighten around the stomach and partially block it (intervention stage). The band can remain in a tightened position for a preset, individualized period of time, at the end of which a signal is sent to deactivate the magnet and loosen the band. The system is then in the stand-by stage ready to activate again according to a subsequent eating detection signal. In various embodiments, the system can include a miniature inclinometer that helps avoid inflation of the band when the patient is in a supine position, which can indicate sleep.

The intervention stage can comprise a start phase and hold phase. The start phase of the intervention comprises activating the magnetic band thus tightening it, and the hold phase of the intervention stage comprises keeping the band tightened. The start phase of the intervention can be quick with a holding period that is variable according to the time need to keep the gastric band in a tightened state. For example, the start phase of the intervention can be about 1, 2, 3, 4, or 5, seconds. In alternative embodiments, start phase of the intervention can be about 5, 10, 15, 20, 25, 30, 35, 40, 45, 50, 55, or 60 seconds. In various embodiments, the holding period can be the length of a meal. In various embodiments, the holding period can be, but is not limited to, 1, 2, 3, 4, 5, 6, 7, 8, 9, 10, 11, 12, 13, 14, 15, 16, 17, 18, 19, or 20 minutes depending on the duration of the meal. Thus, the holding period can be, but is not limited to, 1-5 minutes, 5-10 minutes, 10-15 minutes, 15-20 minutes, 20-25 minutes, 25-30 minutes, 30-40 minutes, 40-50 minutes, 50-60 minutes or longer.

The stand-by stage allows for the gastric band to remain at a loosened state until the intervention stage is started again.

Embodiments also provide for a method of controlling a gastric band. The method can comprise providing a system comprising a gastric band, an eating detection sub-system; and placing the system in a subject. The method can further comprise: detecting food ingestion using the eating detection sub-system; and having the microprocessor initiate the intervention stage. The method can further comprise: detection of the cessation of food ingestion.

Automatic Detection of Eating

The eating detection sub-system can act as a controller of the system. After the detection of eating, an algorithm starts a time-controlled program, with a variable time delay and sends a signal to activate and tighten the magnetic band. The band can remain activated for variable periods of time, which can be adjusted to the eating habits of individual patients. At the end of this preset period of time the band can be deactivated. For example, the system can be set to keep the band activated for 30 min after the detection of eating, and then to deactivate. A subsequent eating detection can again activate the band for another period of 30 min. In a different embodiment the period of activation could be 40, 50 or 60 minutes, or other durations as required per individual eating habits.

The system can also be set to keep the band activated for various periods of time, per individual specifications.

Various embodiments utilize systems and methods of automatic detection of eating to initiate the control of the gastric band. In various embodiments, the automatic detection of eating can utilize the methods, devices, and systems set for the in U.S. Patent Application Publication no. 2010/0076345, which is hereby incorporated by reference as though fully set forth in its entirety.

Electrical activity of the lower esophageal sphincter has been recorded and studied. Swallowing produces changes in the motor activity of the LES. The inventors believed that these changes are related to specific changes in LES electrical activity. The beginning and duration of a meal can be identified by distinct, easily recognizable changes in the amplitude of LES electrical activity. These changes also depend on the type of substance being swallowed (e.g., saliva, liquid and solids), and are most prominent with solid food. Further, during fasting, transient increases in LES electrical activity not related to swallowing do not produce the same increase in electrical activity as seen during swallowing of food. Thus, changes in LES electrical activity can be used for eating detection.

The method of detecting food or drink intake in a subject can comprise: a) placing one or more electrodes in contact with or proximate to the subject's LES; and b) identifying food or drink intake by monitoring electrical activity in or proximate to the LES using one or more electrodes. In various embodiments, bipolar electrodes can be used and thus, only one lead is necessary.

The lower esophageal sphincter is a ring of muscle tissue located at the bottom of the esophagus where the esophagus meets the stomach. Normally, the LES acts as a valve to prevent the backflow of stomach contents into the esophagus. The junction between the esophagus and the stomach is called the gastroesophageal junction.

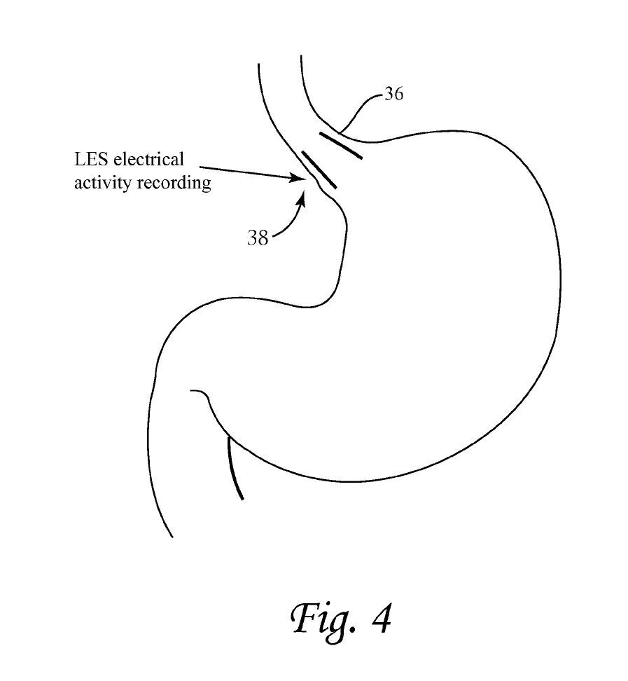

Referring to FIG. 4, in one embodiment, one or more electrodes 38 are placed in contact with the LES 36 or in contact with a proximate region to the LES and the electrical activity is monitored at that location. An increase in the amplitude of electrical activity in the monitored location indicates food or drink intake, and a decrease in the amplitude back to about baseline level indicates the cessation of food or drink intake. Further, the degree of the change in amplitude (e.g., increase in amplitude) can be used to differentiate between types of swallows (saliva, liquid or solid food). The duration of change in amplitude helps to determine the length of period of food consumption. For example, a short duration indicates simple swallows or a very small snack and a long duration indicates the consumption of a larger meal. The electrical activity of the LES while in a resting or non-swallowing state can establish the baseline level, and amplitudes above the baseline can indicate dry swallows, wet swallows, or solid food swallows, depending on the size and duration of the amplitudes.

In some embodiments, a pair of electrodes is placed. Two electrodes can be positioned at opposite sides of the gastroesophageal junction (GEJ). In particular embodiments, one electrode is positioned in the left aspect of the GEJ and a second electrode is positioned in the right aspect of the GEJ.

In other embodiments, one or more electrodes are positioned away from the vagus nerve trunks. In a particular embodiment, one or more electrodes are positioned as far away from the vagus nerve trunks as possible so long as electrical activity indicative of food or drink intake can be detected. In a particular embodiment, two electrodes are positioned as far away from the vagus nerve trunks as possible.

An electrode can be of any size suitable for placement on or in the LES, or on or in a proximate region to the LES. In various embodiments, the electrodes can be about 1 mm long to about 50 mm long, about 5 mm long to about 25 mm long, or about 10 mm long to about 20 mm long. In one embodiment, the electrode can be about 15 mm long. The electrode can also be of any shape suitable for placement on the LES or on a proximate region to the LES; for example, circular, square, rectangular, etc. The electrode can also be of any dimension suitable for placement on the LES or on a proximate region to the LES. The electrode can be attached on the surface of the LES or proximate region, or implanted into the LES or proximate region.

Placing an electrode in contact with the LES or proximate to the LES can be performed by any method known in the art; for example, by a surgical procedure or by an endoscopic procedure. The electrode can be placed on any level in the LES tissue from the inner lining (i.e., mucosa) to the muscle layer. In one particular embodiment, an electrode can be sutured to a muscle layer of the LES or a proximate region to the LES.

In some embodiments, monitoring the electrical activity comprises detecting the electrical activity in the LES. In particular embodiments, monitoring the electrical activity comprise measuring the amplitude and/or duration of the electrical activity in the LES.

An increase in amplitude of the monitored electrical activity to a value greater than baseline amplitude can indicate food or drink intake. In certain embodiments, an about three to about four fold increase in amplitude from baseline amplitude indicates food or drink intake.

In some embodiments, an amplitude of about 0.30 mV to about 0.90 mV indicates a dry swallow, or an amplitude of about 0.40 mV to about 0.80 mV, about 0.45 mV to about 0.75 mV, or about 0.5 mV to about 0.7 mV indicates a dry swallow. In a particular embodiment, an amplitude of about 0.6 mV indicates a dry swallow. Alternatively, an about two-fold increase in amplitude indicates a dry swallow. A “dry swallow” is a swallow in the absence of food or drink.

In some embodiments, an amplitude of about 0.31 mV to about 1.03 mV indicates a drink intake (wet swallow), or an amplitude of about 0.43 mV to about 0.91 mV, about 0.52 mV to about 0.88 mV, or about 0.58 mV to about 0.82 mV indicates a drink intake. In a certain embodiment, an amplitude of about 0.7 mV indicates a drink intake. Alternatively, an about two-fold increase in amplitude indicates a wet swallow.

In some embodiments, an amplitude of about 0.55 mV to about 1.57 mV indicates solid food intake, or an amplitude of about 0.72 mV to about 1.4 mV, about 0.81 mV to about 1.32 mV, or about 0.89 mV to about 1.23 mV indicates solid food intake. In a particular embodiment, an about 1.06 mV indicates solid food intake. Alternatively, a greater than three-fold increase in amplitude indicates solid food intake, or an about three to about four fold increase in amplitude indicates solid food intake.

The specific amplitudes indicative of dry swallows, wet swallows and food intake will vary depending on the subject being examined. The range of amplitudes for a specific subject can be obtained by measuring the subject's background level of electrical activity while the subject is in a resting or non-swallowing state, then measuring the amplitudes when the subject is performing a dry swallow, is swallowing liquid, and is swallowing solid food. These observed amplitudes can be used to identify background electrical activity and different types of swallows when the subject is subsequently monitored for food or drink intake.

Reversion of an increased amplitude back to baseline or to a value of approximately baseline amplitude can indicate that food or drink intake has stopped. Further, a decrease in amplitude from a higher value to a lower value can indicate that food or drink intake has stopped. In some embodiments, an about three to about four fold decrease in amplitude from the increased amplitude indicates food or drink intake has stopped. In certain embodiments, an amplitude of about 0.135 mV to about 0.495 mV indicates that food or drink intake has stopped, or an amplitude of about 0.195 mV to about 0.435 mV, about 0.225 mV to about 0.405 mV, or about 0.255 mV to about 0.375 mV indicates that food or drink intake has stopped. In a particular embodiment, an amplitude of about 0.315 mV indicates that food or drink intake has stopped.

Data on electrical activity in the LES can be transmitted to a recording/analyzing device, such as a microprocessor incorporated in the system, by way of electrodes. In another embodiment, a miniaturized recorder implanted in the LES or in contact with a proximate region to the LES can transmit data in a wireless fashion to an implanted system, or to an outside device.

In some embodiments, a signal indicating that a subject has started consuming food or drink, is in the process of consuming food or drink, has stopped consuming food or drink, has consumed food or drink, or any combination thereof, can be generated based on the amplitude and duration of the electrical activity of the LES or proximate to the LES. The signal can be sent to a receiving device, such as a microprocessor in the computerized hydraulic system or a system containing a receiving device, or other device or system associated with food or drink intake or the cessation of food or drink intake. As such, additional embodiments can further comprise using a receiver to receive signals regarding the subject's food or drink intake. In particular embodiments, the receiving device is used in a clinical application associated with food or drink intake. Thus, the detection of food or drink intake or cessation of food or drink intake, or signals indicative thereof, may be used in conjunction with other technology for clinical applications. That is, the detection of food or drink intake or the cessation of food or drink intake, or signals indicative thereof, can be used to trigger an intervention treatment that is associated with the food or drink intake or the cessation of food or drink intake.

Additional embodiments can further comprise using a computer or computer system to perform a number of functions, for example, including but not limited to receiving electrical signals, analyzing electrical signals, processing electrical signals, and sending a signal regarding the received, analyzed and/or processed electrical signals to another system, computer or device. Such computers and computer systems are known in the art and one of skill in the art will be able to determine, without undue experimentation, a computer or a computer system that is suitable for such use.

A device for practicing the method of detecting food or drink intake can comprise: a) one or more electrodes, for monitoring electrical activity of the subject's LES or a region proximate to the LES; and a microprocessor that can monitor electrical activity and incorporates an algorithm that detect eating and generate electrical signals based on the monitored electrical activity. Such microprocessor can be part of a computerized hydraulic system The device can further comprise a recording module, for recording electrical data based on the monitored electrical activity. In some embodiments, one or more pairs of electrodes is utilized. The detection device can be configured to automatically detect food or drink intake in a subject.

In one embodiment, the detection device comprises one or more electrodes, and a microprocessor, wherein the one or more electrodes are connected to the microprocessor that can analyze electrical signals from the LES or a region proximate to the LES and also send electrical signals to activate the hydraulic system. The one or more electrodes can be one or more pairs of electrodes, or be a single lead (e.g., bipolar electrode). In particular embodiments, the detection device is configured to measure the amplitude and/or duration of the electrical activity in the LES or in the proximate region to the LES. In some embodiments, the detection device is an implantable device.

In one embodiment the whole system is implantable. In another embodiment, the microprocessor is positioned outside the body, and both recording of electrical signals from the LES or a region proximate to the LES and delivery of signals to the magnetic band can be done by wireless connections. In some embodiments, a recording of the electrical activity is obtained by placing wands on the subject's skin that detect the electrical activity, and connecting the wands to data loggers.

The electrode can be any size suitable for placement on the LES or a proximate region to the LES. In various embodiments, the electrodes can be about 1 mm long to about 50 mm long, about 5 mm long to about 25 mm long, or about 10 mm long to about 20 mm long. In one embodiment, the electrode may be about 15 mm long. The electrode can be any shape suitable for placement at the LES; for example, circular, square, rectangular, etc., and can be any dimension suitable for placement at the LES.

In some embodiments, the detection device can further comprise a computer. The computer can be used to perform a number of functions; for example, including but not limited to receiving electrical signals, analyzing electrical signals, processing electrical signals, and sending a signal regarding the received, analyzed and/or processed electrical signals to another system, computer or device.

In one embodiment, the detection device is configured to generate and send a signal to another device indicating the electrical activity of the LES. In some embodiments, the signal can be a signal that indicates that the subject has started consuming food or drink, is consuming food or drink, has stopped consuming food or drink, has consumed food or drink, or any combination thereof.

In some embodiments, the detection device is configured to generate and send a signal when an increase in amplitude from baseline amplitude is detected. In another embodiment, the device is configured to generate and send a signal that the subject has consumed food or drink when an about three to about four fold increase in amplitude from a baseline amplitude is detected.

In some embodiments, the detection device is configured to generate and send a signal that the subject has swallowed when an amplitude of about 0.30 mV to about 0.90 mV has been detected. In particular embodiments, the device is configured to generate and send a signal that the subject has swallowed when an amplitude of 0.40 mV to about 0.80 mV, about 0.45 mV to about 0.75 mV, or about 0.5 mV to about 0.7 mV has been detected. In a particular embodiment, the device is configured to generate and send a signal that the subject has swallowed when an amplitude of about 0.6 mV has been detected. Alternatively, the device is configured to generate and send a signal that the subject has swallowed when an about two-fold increase in amplitude has been detected.

In some embodiments, the detection device is configured to generate and send a signal that the subject has consumed a liquid when an amplitude of about 0.31 mV to about 1.03 mV, about 0.43 mV to about 0.91 mV, about 0.52 mV to about 0.88 mV, or about 0.58 mV to about 0.82 mV has been detected. In a particular embodiment, the device is configured to generate and send a signal that the subject has consumed a liquid when an amplitude of about 0.7 mV has been detected. Alternatively, the device is configured to generate and send a signal that the subject has consumed a liquid when an about two-fold increase in amplitude has been detected.

In some embodiments, the detection device is configured to generate and send a signal that the subject has consumed solid food when an amplitude of 0.55 mV to about 1.57 mV has been detected. In certain embodiments, the device is configured to generate and send a signal that the subject has consumed food when amplitude of about 0.72 mV to about 1.4 mV, about 0.81 mV to about 1.32 mV, or about 0.89 mV to about 1.23 mV has been detected. In a particular embodiment, the device is configured to generate and send a signal that the subject has consumed food when amplitude of about 1.06 mV has been detected. Alternatively, the device is configured to generate and send a signal that the subject has consumed food when greater than a two-fold increase in amplitude has been detected. In particular embodiments, the device is configured to generate and send a signal that the subject has consumed food when an about three to about four fold increase in amplitude is detected.

Reversion of an increased amplitude back to baseline or to a value of approximately baseline amplitude can indicate that food or drink intake has stopped. Further, a decrease in amplitude from a higher value to a lower value can indicate that food or drink intake has stopped. In some embodiments, the detection device is configured to generate and send a signal that the subject has ceased consuming food or drink when a reversion of the increased amplitude back to approximately baseline amplitude is detected. In certain embodiments, the device is configured to generate and send a signal that the subject has stopped consuming food or drink when an about three to about four fold decrease in amplitude from the increased amplitude is detected.

In some embodiments, the detection device can be configured to generate and send a signal that the subject has ceased consuming food or drink when an amplitude of about 0.135 mV to about 0.495 mV has been detected. In particular embodiments, the device may be configured to generate and send a signal that the subject has ceased consuming food or drink when amplitude of about 0.195 mV to about 0.435 mV, about 0.225 mV to about 0.405 mV, about 0.255 mV to about 0.375 mV has been detected. In a particular embodiment, the device is configured to generate and send a signal that the subject has ceased consuming food when an amplitude of about 0.315 mV has been detected.

These signals may be useful for a variety of clinical applications. The signals may be used in conjunction with other technology for clinical applications. That is, the signal generated when food or drink intake is detected or when the cessation of food or drink intake is detected may be used to trigger an intervention treatment that is associated with the food or drink intake or the cessation of food or drink intake.

A system for practicing the method can comprise the following. A pair of electrodes is implanted in the lower esophageal sphincter (LES) at the level of the gastro-esophageal junction. The electrodes are connected to a microprocessor. The microprocessor receives and processes signals from the electrodes regarding the subject's intake of food and drink. The microprocessor can send a signal to a pump. All of these components can be manufactured separately, in combination, or as a single device.

In one embodiment, the system comprises a device for monitoring the electrical activity of the LES and a computer for interpreting and/or recording the electrical activity of the LES. In another embodiment, the system further comprises a device for recording the electrical activity of the LES. The device for monitoring the electrical activity can comprise one or more electrodes, and a microprocessor, wherein the microprocessor, can be connected to the one or more electrodes and the device is configured to measure the electrical activity in the LES or in a proximate region to the LES. In particular embodiments, the device for monitoring the electrical activity is configured to measure the amplitude and/or duration of the electrical activity in the LES or in the proximate region to the LES. The pulse generator can be used to generate pulses or signals that are read and processed by a computer.

The electrode can be any size suitable for placement at the LES. In various embodiments, the electrode can be about 1 mm long to about 50 mm long, about 5 mm long to about 25 mm long, or about 10 mm long to about 20 mm long. In one embodiment, the electrode is about 15 mm long. The electrode can be any shape suitable for placement at the LES, such as circular, square, rectangular, etc. The electrode can also be of any dimension suitable for placement at the LES.

A computer can be used to perform a number of functions, for example, including but not limited to receiving electrical signals, analyzing electrical signals, processing electrical signals, and sending a signal regarding the electrical signals to another system, computer or device

Additional embodiments of the system further comprise a receiver for receiving signals regarding a subject's food or drink intake.

The system can comprise a device for monitoring the electrical activity and a device for sending a signal to a second system or device. In one embodiment, the second system or device is a system or device for the treatment of obesity.

The device for sending a signal to a second system or device can be configured to generate and send a signal to indicate the electrical activity of the LES. In particular embodiments, the signal is a signal indicating that the subject has started consuming food or drink, is in the process of consuming food or drink, has stopped consuming food or drink, has consumed food or drink, or any combination thereof.

In some embodiments, the device for sending a signal is configured to generate and send a signal when an increase in amplitude from baseline amplitude is detected. In certain embodiments, the device is configured to generate and send a signal that the subject has consumed food or drink when an about three to about four fold increase in amplitude from a baseline amplitude is detected.

In some embodiments, the device is configured to generate and send a signal that the subject has swallowed when an amplitude of about 0.30 mV to about 0.90 mV has been detected. In particular embodiments, the device is configured to generate and send a signal that the subject has swallowed when an amplitude of 0.40 mV to about 0.80 mV, about 0.45 mV to about 0.75 mV, or about 0.5 mV to about 0.7 mV has been detected. In a particular embodiment, the device is configured to generate and send a signal that the subject has swallowed when an amplitude of about 0.6 mV has been detected. Alternatively, the device is configured to generate and send a signal that the subject has swallowed when an about two-fold increase in amplitude has been detected.

In some embodiments, the device is configured to generate and send a signal that the subject has consumed a liquid when amplitude of about 0.31 mV to about 1.03 mV has been detected. In other embodiments, the device is configured to generate and send a signal that the subject has consumed a liquid when amplitude of 0.43 mV to about 0.91 mV, about 0.52 mV to about 0.88 mV, or about 0.58 mV to about 0.82 mV has been detected. In a particular embodiment, the device is configured to generate and send a signal that the subject has consumed a liquid when amplitude of about 0.7 mV has been detected. Alternatively, the device is configured to generate and send a signal that the subject has consumed a liquid when an about two-fold increase in amplitude has been detected.

In some embodiment, the device is configured to generate and send a signal that the subject has consumed food when an amplitude of 0.55 mV to about 1.57 mV has been detected. In other embodiments, the device is configured to generate and send a signal that the subject has consumed food when an amplitude of about 0.72 mV to about 1.4 mV, about 0.81 mV to about 1.32 mV, or about 0.89 mV to about 1.23 mV has been detected. In a particular embodiment, the device is configured to generate and send a signal that the subject has consumed food when an amplitude of about 1.06 mV has been detected. Alternatively, the device is configured to generate and send a signal that the subject has consumed food when greater than a two-fold increase in amplitude has been detected. In particular embodiments, the device is configured to generate and send a signal that the subject has consumed food when an about three to about four fold increase in amplitude is detected.

Reversion of an increased amplitude back to baseline or to a value of approximately baseline amplitude can indicate that food or drink intake has stopped. Further, a decrease in amplitude from a higher value to a lower value can indicate that food or drink intake has stopped. In some embodiments, the device is configured to generate and send a signal that the subject has ceased consuming food or drink when a reversion of the increased amplitude back to approximately baseline amplitude is detected. In particular embodiments, the device is configured to generate and send a signal that the subject has stopped consuming food or drink when an about three to about four fold decrease in amplitude from the increased amplitude is detected.

In some embodiment, the device is configured to generate and send a signal that the subject has ceased consuming food or drink when an amplitude of about 0.135 mV to about 0.495 mV has been detected. In certain embodiments, the device is configured to generate and send a signal that the subject has ceased consuming food or drink when an amplitude of about 0.195 mV to about 0.435 mV, about 0.225 mV to about 0.405 mV, about 0.255 mV to about 0.375 mV has been detected. In a particular embodiment, the device is configured to generate and send a signal that the subject has ceased consuming food when an amplitude of about 0.315 mV has been detected.

Various embodiments are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments have been shown and described, it will be obvious to those skilled in the art that, based upon the teachings herein, changes and modifications may be made without departing from this invention and its broader aspects. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

What is claimed is:

1. A method for controlling a band implanted within a subject and around the subject's stomach, comprising
adjusting the band in response to changes in electrical activity of the subject's lower esophageal sphincter.

2. The method of claim 1, further comprising monitoring the changes in electrical activity prior to adjusting the band.

3. The method of claim 1, wherein the adjusting comprises tightening or loosening the band around the subject's stomach.

4. The method of claim 1, wherein the band is activated when eating is indicated by the changes in electrical activity, and is deactivated at preset times after being activated.

5. The method of claim 1, wherein the adjusting comprises an intervention stage.

6. The method of claim 5, wherein the intervention stage comprises a start phase, hold phase and stand-by stage.

7. The method of claim 6, wherein the start phase of the intervention comprises activating the magnetic band thus tightening it, and the hold phase of the intervention stage comprises keeping the band tightened.

8. The method of claim 6, wherein the start phase of the intervention can be about 1 to 60 seconds.

9. The method of claim 6, wherein the hold phase of the intervention can be about 1 to 60 minutes.

10. The method of claim 1, wherein the band is adjusted using electromagnetic force.

11. The method of claim 1, wherein the changes in the electrical activity indicate that the subject has started consuming food or drink, is consuming food or drink, has stopped consuming food or drink, has consumed food or drink, or any combination thereof.

12. The method of claim 1, wherein said changes in electrical activity comprise changes in electrical amplitude or duration.

13. The method of claim 12, wherein an increase in amplitude to a value greater than baseline indicates food or drink intake.

14. The method of claim 12, wherein an about three to about four fold increase in amplitude from baseline indicates food or drink intake.

15. The method of claim 12, wherein an amplitude of about 0.30 mV to about 0.90 mV, or about two-fold increase in amplitude from baseline, indicates a dry swallow.

16. The method of claim 12, wherein an amplitude of about 0.31 mV to about 1.03 mV, or an about two-fold increase in amplitude from baseline, indicates a wet swallow.

17. The method of claim 12, wherein an amplitude of about 0.55 mV to about 1.57 mV, or a greater than three-fold increase in amplitude from baseline, or an about three to about four fold increase in amplitude from baseline, indicates solid food intake.

18. The method of claim 12, wherein an amplitude of about 0.55 mV to about 1.57 mV, or a greater than three-fold increase in amplitude from baseline, or an about three to about four fold increase in amplitude from baseline, indicates solid food intake.

19. The method of claim 1, wherein the subject is undergoing treatment for obesity, treatment to prevent obesity, or treatment for diabetes, or any combination thereof.

20. The method of claim 1, wherein the band comprises
two jaws hinged by a rotating axis on one side of the jaws; and
two electromagnets on the other side of the jaws, one electromagnet for closing the jaws, the other electromagnet for keeping the jaws closed.

21. The method of claim 1, wherein the band comprises
a plurality of electromagnetic segments arranged end-to-end in a series, each segment comprising a rod connected to an electromagnet; and
a locking electromagnetic segment connected to one or both ends of the series.

22. A system for a subject, comprising a monitoring unit that monitors electrical activity changes of the subject's lower esophageal sphincter; and an adjustable band implantable within the subject and around the subject's stomach and that responds to signals from the monitoring unit about the electrical activity changes.

23. The system of claim 22, wherein the monitoring unit comprises a microprocessor that monitors electrical activity and detects eating and/or generates electrical signals based on the monitored electrical activity.

24. The system of claim 22, further comprising a recording module for recording electrical data based on the monitored electrical activity.

25. The system of claim 22, further comprising a pulse generator and a recording module.

26. The system of claim 22, further comprising a computer for receiving electrical signals, analyzing electrical signals, processing electrical signals, and sending a signal regarding the electrical signals to another system, computer or device.

27. The system of claim 22, individualized for each subject.

28. The system of claim 22, further comprising one or more electrodes for detecting the electrical activity changes and positionable within, in contact with or proximate to the gastroesophageal junction of the subject.

29. The system of claim 22, wherein the band comprises
two jaws hinged by a rotating axis on one side of the jaws; and
two electromagnets on the other side of the jaws, one electromagnet for closing the jaws, the other electromagnet for keeping the jaws closed.

30. The system of claim 22, wherein the band comprises
a plurality of electromagnetic segments arranged end-to-end in a series, each segment comprising a rod connected to an electromagnet; and
a locking electromagnetic segment connected to one or both ends of the series.

31. A band implantable within a subject and around the subject's stomach, comprising
two jaws hinged by a rotating axis on one side of the jaws; and
two electromagnets on the other side of the jaws, one electromagnet for closing the jaws, the other electromagnet for keeping the jaws closed.

32. A band implantable within a subject and around the subject's stomach, comprising
a plurality of electromagnetic segments arranged end-to-end in a series, each segment comprising a rod connected to an electromagnet; and
a locking electromagnetic segment connected to one or both ends of the series.
